(19) United States
(12) Patent Application Publication
(10) Pub. No.: US 2007/0218455 A1
(43) Pub. Date: Sep. 20, 2007

(54) Sample preparation of biological cells for analysis

(75) Inventors: Mark G. Knize, Tracy, CA (US); Kristen S. Kulp, Livermore, CA (US); Kuang Jen J. Wu, Cupertino, CA (US)

Correspondence Address: Eddie E. Scott;Assistant Laboratory Counsel, Lawrence Livermore national laboratory, P.O. Box 808, L-703, Livermore, CA, 94551, US

(73) Assignee: The Regents of the University of California

(21) Appl. No.: 11/378,853

(22) Filed: Mar. 16, 2006

(51) Int. Cl. C12Q 1/00; C12M 3/00

(52) U.S. Cl. 435/4; 435/287.1

(57) ABSTRACT

A system for reliably and reproducibly exposing the contents of a biological cell to an analysis instrument comprising a sample handling unit that includes a first substrate with first substrate face and a second substrate with second substrate face. The first substrate face and the second substrate face are opposed. The biological cell is positioned between the first substrate face and the second substrate face. A framework with a mechanism for applying a range of pressures to the first substrate face and the second substrate face reliably and reproducibly exposes the contents of the biological cell to the analysis instrument.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to sample preparation and more particularly to sample preparation of biological cells for use with imaging mass spectrometry.

2. State of Technology

The article “Identification of Cellular Sections with Imaging Mass Spectrometry Following Freeze Fracture,” by Thomas P. Roddy et al, in Analytical Chemistry, Vol. 74, No. 16, pages 4020-4026, Aug. 15, 2002, provides the following state of technology information: “A sandwich fracture technique used in EM has been modified for TOF-SIMS and ion microscopy. In this technique, cells in aqueous media and spacer beads of cellular dimensions are frozen between two silicon wafers. The spacer beads prevent crushing of the cells and influence the propagation of cleavage planes through the ice. In addition, cellular morphology, ice crystallization, and cellular-surface adhesion forces influence which section of the cell is exposed during freeze fracture. In scanning electron microscopy (SEM), a highly detailed morphological view of a metal-coated fractured sample is obtained.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a system of reliably and reproducibly exposing the contents of a biological cell to an analysis instrument. The system comprises growing upon or applying the biological cell to a first substrate, positioning a second substrate on top of the first substrate and the biological cell, controllable and reproducibly applying pressure to the first substrate and the second substrate to flatten the biological cell exposing the contents of the biological cell to the analysis instrument. The present invention also provides an apparatus for reliably and reproducibly exposing the contents of a biological cell to an analysis instrument. The apparatus comprises a sample handling unit. The sample handling unit includes a first substrate with first substrate face and a second substrate with second substrate face. The first substrate face and the second substrate face are opposed. The biological cell is positioned between the first substrate face and the second substrate face. A framework with a mechanism for applying a range of pressures to the first substrate face and the second substrate face reliably and reproducibly exposes the contents of the biological cell to the analysis instrument.

The present invention exposes the intracellular contents of single biological cells by controlled crushing thereby providing a method to reproducibly open the interior of the cells for analysis. Exposing the cell contents in a way that maintains the chemical integrity of the intracellular environment is necessary for single cell analysis. Secondary ion mass spectrometry, a technique that produces a chemical map of a biological sample, is limited to analyzing the top few molecules of the surface layer of an object.

The existing method for exposing the inner surfaces of cells is to freeze the cells between two silicon chips and then pull the chips apart, ripping open the cells in some areas. This is a haphazard and unreliable art resulting in a few usable cells from thousands under study. The cells that are fractured have uncertain fracture planes, many times only exposing inner cell membranes and not the desired cell interior. The present invention overcomes these disadvantages by reproducibly exposing the cell contents of all of the cells under study.

The present invention can be used in any instance where there is a need to prepare biological samples while maintaining the molecular integrity of the samples. There is a current emphasis in biology on the need for early detection and diagnosis of disease to maximize the effectiveness of treatment. In many cases, identifying and characterizing rare cells that appear in easily accessible fluids like blood, saliva, nipple aspirate fluid, and urine can facilitate early detection. This invention can supply reliable sample preparation that will enable these rare cells to be mass-imaged for the first time. In research laboratories, there is a need for sample preparation in experiments with rare stem cells, cultured cells or for tissue samples. This device will greatly improve the effectiveness of sample preparation in the growing field of biological mass spectrometry.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 illustrates freeze fracture/freeze etch of the prior art.

FIG. 2 illustrates a system constructed in accordance with the present invention.

FIG. 3 shows the sample handling unit of FIG. 2 in greater detail.

FIG. 4 illustrates another system constructed in accordance with the present invention.

FIG. 5 shows the sample handling unit of FIG. 4 in greater detail.

FIG. 6 illustrates another system constructed in accordance with the present invention.

FIG. 7 shows the sample handling unit of FIG. 6 in greater detail.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

Although imaging mass spectrometry is a well-established material science technique, the potential to image and characterize biological samples has not yet been fully explored, in part due to the complexities inherent in biological samples. Virtually all the intracellular machinery is contained within cell membranes that are barriers to analysis by static surface analysis instruments like secondary ion mass spectrometers. For instruments that can penetrate through the cell surface, cellular damage from the analysis can affect the results of the experiment. One approach to opening the cells, while preserving the molecular integrity, is the freeze-fracture technique. With this technique the cells are frozen between two silicon chips and then pull the chips are pulled apart, ripping open the cells in some areas. This opens only a fraction of the cells and leaves them fractured along random fracture planes.

An example of Freeze Fracture is described in the article “Identification of Cellular Sections with Imaging Mass Spectrometry Following Freeze Fracture,” by Thomas P. Roddy et al, in Analytical Chemistry, Vol. 74, No. 16, pages 420-4026, Aug. 15, 2002 as follows, “a sandwich fracture technique used in EM has been modified for TOF-SIMS and ion microscopy. In this technique, cells in aqueous media and spacer beads of cellular dimensions are frozen between two silicon wafers. The spacer beads prevent crushing of the cells and influence the propagation of cleavage planes through the ice. In addition, cellular morphology, ice crystallization, and cellular-surface adhesion forces influence which section of the cell is exposed during freeze fracture. In scanning electron microscopy (SEM), a highly detailed morphological view of a metal-coated fractured sample is obtained.” The article “Identification of Cellular Sections with Imaging Mass Spectrometry Following Freeze Fracture,” by Thomas P. Roddy et al, in Analytical Chemistry, Vol. 74, No. 16, pages 420-4026, Aug. 15, 2002 is incorporated herein by this reference.

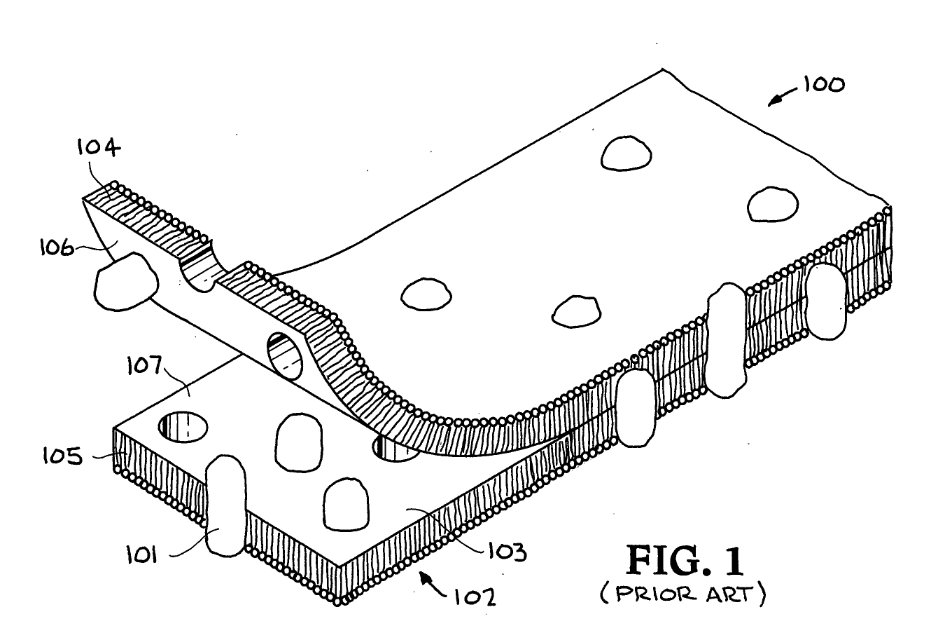

Referring now to the drawings, and in particular FIG. 1, an electron microscope view of membranes via freeze fracture/freeze etch of the prior art is illustrated. The view is designated generally by the reference numeral 100. The FIG. 1 illustration shows a freeze-fracture/freeze etch view. It can best see protein distribution via a technique called freeze fracture/freeze etch 102. The freeze-fracture/freeze etch technique starts with rapid freezing of a cell. Then the frozen cells are cleaved along a fracture plane 103. This fracture plane 103 is in between the leaflets 104 and 105 of the lipid bilayer. The two fractured sections, e-face 106 and p-face 107 are then coated with heavy metal (etched) and a replica is made of their surfaces. The organization or structure of the transmembrane proteins 101 can often be visualized. This replica can be viewed in an electron microscope. Homogeneous regions are seen where there was only the exposed lipid leaflet. In certain areas of the cell, protrusions or bumps are seen. Sometimes structure within the bumps themselves can be seen. These are the membrane proteins.

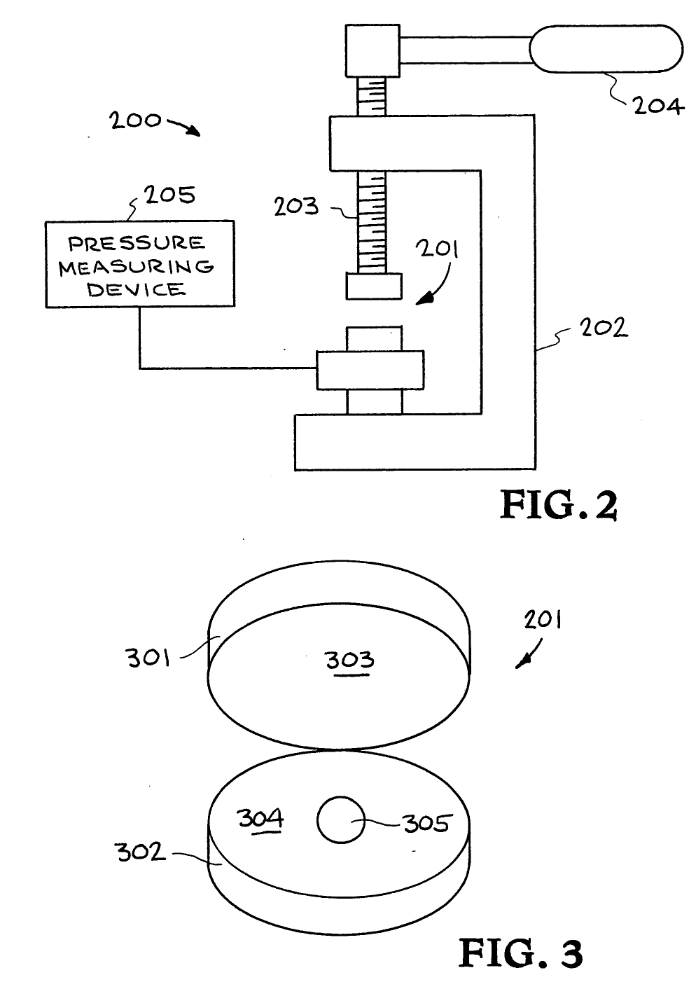

Referring now to FIG. 2, a system constructed in accordance with the present invention is illustrated. The system is designated generally by the reference numeral 200. The system 200 reliably and reproducibly exposes a biological cell contents to an analysis instrument. The system 200 includes a sample handling unit 201. The sample handling unit 201 will be described in greater detail subsequently. The sample handling unit 201 has two parallel faces to hold biological cells.

A framework 202 with a mechanical mechanism to apply a range of pressures is connected to the sample handling unit 201. The mechanical mechanism includes a screw device 203 operated by a handle 204 to controllably and reproducibly apply a range of pressures to the sample handling unit 201. A pressure measuring device 205 is connected to the sample handling unit 201. The pressure measuring device 205 enables reproducible pressure application to the sample handling unit 201. The framework 202, mechanical mechanism, and sample handling unit 201 may be cooled to reduce chemical decomposition of the sample.

The system 200 provides an apparatus and method to reliably and reproducibly expose the cell contents to an analysis instrument. This will facilitate: 1) experiments on a single cells, 2) experiments for which only a few cells are available, or 3) enable all cells to be analyzed when many cells are present. The system 200 will reduce the analysis time needed to locate properly prepared cells on a substrate, and eliminate the questions about whether the cell interior is exposed to the analysis beam. These changes will reduce the costs of sample preparation and analysis time, and enable analysis of rare human cells that may be important for detecting and diagnosing disease.

Referring now to FIG. 3, the sample handling unit 201 is illustrated in greater detail. The sample handling unit 201 includes a first substrate 301 and a second substrate 302 immediately below substrate 301. The first substrate 301 and the second substrate 302 are made of materials such as silicon, stainless steel, etc. The first substrate 301 has a face 303 and the second substrate 302 has a face 304 directly opposed to the face 303 on the first substrate 301. A biological cell 305 is positioned between the face 304 on the second substrate 302 and the face 303 on the first substrate 301. It is to be understood that a single biological cell 305 is shown for illustrative purposes and that multiple biological cells are contemplated by the system 200.

The structural details of the system 200 for reliably and reproducibly exposing the cell 305 contents to an analysis instrument having been described, the operation of the system 200 will now be considered. The biological cell 305 is grown upon or applied to the face 304 on the second substrate 302. The first substrate 301 is applied on top of the biological cell 305. Pressure is applied to the face 304 on the second substrate 302 and the face 303 on the first substrate 301 by the framework 202, mechanical mechanism, and sample handling unit 201 for varying periods of time to flatten the biological cell 305 and release intracellular contents. The pressure measuring device 205 enables reproducible pressure application to the sample handling unit 201. The biological cell 305 is released and surfaces of the biological cell 305 can be analyzed for cell contents.

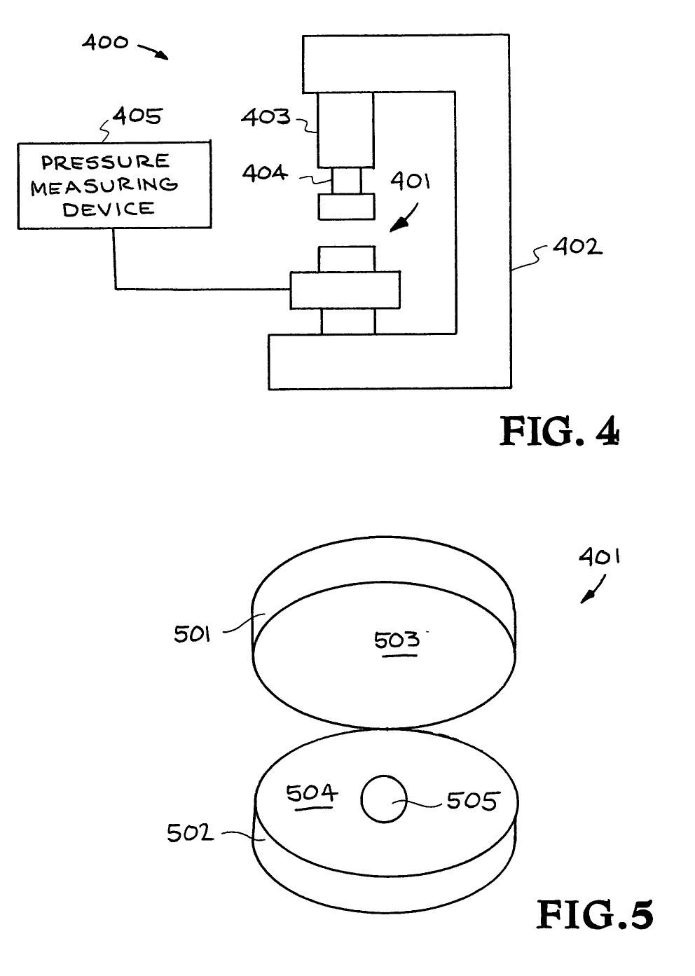

Referring now to FIG. 4, another embodiment of a system constructed in accordance with the present invention is illustrated. This embodiment is designated generally by the reference numeral 400. The system 400 reliably and reproducibly exposes a biological cell contents to an analysis instrument. The system 400 includes a sample handling unit 401. The sample handling unit 401 will be described in greater detail subsequently. The sample handling unit 401 has two parallel faces to hold biological cells.

A framework 402 with a hydraulic mechanism to apply a range of pressures is connected to the sample handling unit 401. The hydraulic mechanism includes a hydraulic cylinder 403 with a piston 404 for controllably and reproducibly apply a range of pressures to the sample handling unit 401. A pressure measuring device 405 is connected to the sample handling unit 401. The pressure measuring device 405 enables reproducible pressure application to the sample handling unit 401. The framework 402, mechanical mechanism, and sample handling unit 401 may be cooled to reduce chemical decomposition of the sample.

The system 400 provides an apparatus and method for reliably and reproducibly expose the cell contents to an analysis instrument. This will facilitate: 1) experiments on a single cells, 2) experiments for which only a few cells are available, or 3) enable all cells to be analyzed when many cells are present. The system 400 will reduce the analysis time needed to locate properly prepared cells on a substrate, and eliminate the questions about whether the cell interior is exposed to the analysis beam. These changes will reduce the costs of sample preparation and analysis time, and enable analysis of rare human cells that may be important for detecting and diagnosing disease.

Referring now to FIG. 5, the sample handling unit 401 is illustrated in greater detail. The sample handling unit 401 includes a first substrate 501 and a second substrate 502 immediately below substrate 501. The first substrate 501 and the second substrate 502 are made of materials such as silicon, stainless steel, etc. The first substrate 501 has a face 503 and the second substrate 502 has a face 504 directly opposed to the face 503 on the first substrate 501. A biological cell 505 is positioned between the face 504 on the second substrate 502 and the face 503 on the first substrate 501. It is to be understood that a single biological cell 505 is shown for illustrative purposes and that multiple biological cells are contemplated by the system.

The structural details of a system for reliably and reproducibly exposing the cell 505 contents to an analysis instrument having been described, the operation of the system will now be considered. The biological cell 505 is grown upon or applied to the face 504 on the second substrate 502. The first substrate 501 is applied on top of the biological cell 505. Pressure is applied to the face 504 on the second substrate 502 and the face 503 on the first substrate 501 by the framework 202, hydraulic mechanism, and sample handling unit 401 for varying periods of time to flatten the biological cell 505 and release intracellular contents. The pressure measuring device 205 enables reproducible pressure application to the sample handling unit 401. The biological cell 505 is released and surfaces of the biological cell 505 can be analyzed for cell contents.

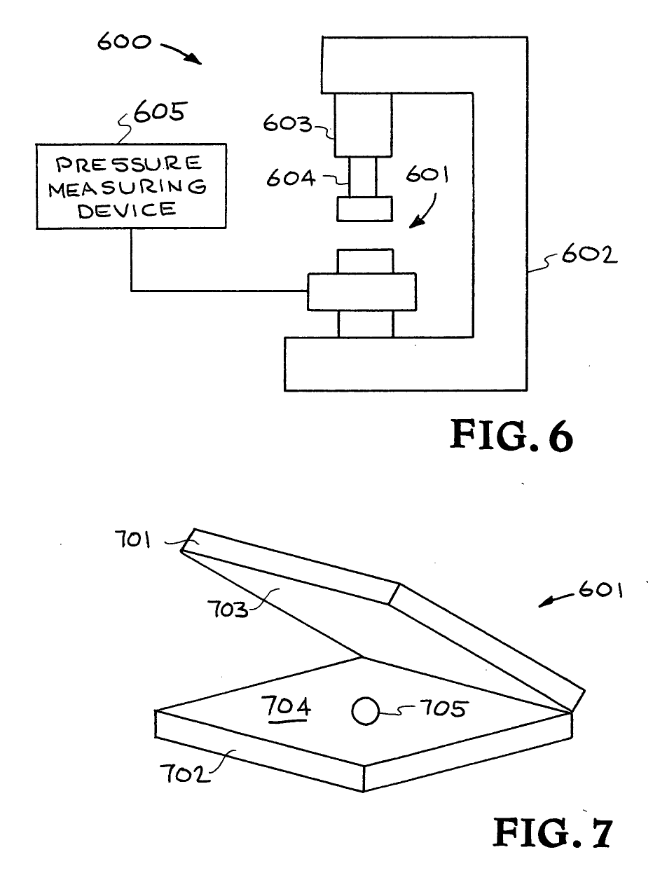

Referring now to FIG. 6, another embodiment of a system constructed in accordance with the present invention is illustrated. This embodiment is designated generally by the reference numeral 600. The system 600 reliably and reproducibly exposes a biological cell contents to an analysis instrument. The system 600 includes a sample handling unit 601. The sample handling unit 601 will be described in greater detail subsequently. The sample handling unit 601 has two parallel faces to hold biological cells.

A framework 602 with an electrical mechanism to apply a range of pressures is connected to the sample handling unit 601. The electrical mechanism includes a solenoid 603 with an arm 604 for controllably and reproducibly apply a range of pressures to the sample handling unit 601. A pressure measuring device 605 is connected to the sample handling unit 601. The pressure measuring device 605 enables reproducible pressure application to the sample handling unit 601. The framework 602, mechanical mechanism, and sample handling unit 601 may be cooled to reduce chemical decomposition of the sample.

The system 600 provides an apparatus and method for reliably and reproducibly expose the cell contents to an analysis instrument. This will facilitate: 1) experiments on a single cells, 2) experiments for which only a few cells are available, or 3) enable all cells to be analyzed when many cells are present. The system 600 will reduce the analysis time needed to locate properly prepared cells on a substrate, and eliminate the questions about whether the cell interior is exposed to the analysis beam. These changes will reduce the costs of sample preparation and analysis time, and enable analysis of rare human cells that may be important for detecting and diagnosing disease.

Referring now to FIG. 7, the sample handling unit 601 is illustrated in greater detail. The sample handling unit 601 includes a first substrate 701 and a second substrate 702 immediately below substrate 701. The first substrate 701 and the second substrate 702 are made of materials such as silicon, stainless steel, etc. The first substrate 701 has a face 703 and the second substrate 702 has a face 704 directly opposed to the face 703 on the first substrate 701. A biological cell 705 is positioned between the face 704 on the second substrate 702 and the face 703 on the first substrate 701. It is to be understood that a single biological cell 705 is shown for illustrative purposes and that multiple biological cells are contemplated by the system.

The structural details of a system for reliably and reproducibly exposing the cell 705 contents to an analysis instrument having been described, the operation of the system will now be considered. The biological cell 705 is grown upon or applied to the face 704 on the second substrate 702. The first substrate 701 is applied on top of the biological cell 705. Pressure is applied to the face 704 on the second substrate 702 and the face 703 on the first substrate 701 by the framework 602, hydraulic mechanism, and sample handling unit 601 for varying periods of time to flatten the biological cell 705 and release intracellular contents. The pressure measuring device 605 enables reproducible pressure application to the sample handling unit 601. The biological cell 705 is released and surfaces of the biological cell 705 can be analyzed for cell contents.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. An apparatus for reliably and reproducibly exposing the contents of a biological cell to an analysis instrument, comprising:
a sample handling unit, said sample handling unit including
a first substrate with first substrate face and
a second substrate with second substrate face, wherein said first substrate face and said second substrate face are opposed and wherein the biological cell is positioned between said first substrate face and said second substrate face, and
a framework with a mechanism for applying a range of pressures to said first substrate face and said second substrate face to reliably and reproducibly exposing the contents of the biological cell to the analysis instrument.

2. The apparatus for reliably and reproducibly exposing the contents of a biological cell to an analysis instrument of claim 1 wherein said mechanism for applying a range of pressures to said first substrate face and said second substrate face is a mechanical mechanism.

3. The apparatus for reliably and reproducibly exposing the contents of a biological cell to an analysis instrument of claim 1 wherein said mechanism for applying a range of pressures to said first substrate face and said second substrate face is a hydraulic mechanism.

4. The apparatus for reliably and reproducibly exposing the contents of a biological cell to an analysis instrument of claim 1 wherein said mechanism for applying a range of pressures to said first substrate face and said second substrate face is an electrical mechanism.

5. The apparatus for reliably and reproducibly exposing the contents of a biological cell to an analysis instrument of claim 1 wherein said mechanism for applying a range of pressures to said first substrate face and said second substrate face is mechanical mechanism or a hydraulic mechanism or an electrical mechanism or a combination of said mechanical mechanism or said hydraulic mechanism or said electrical mechanism.

6. The apparatus for reliably and reproducibly exposing the contents of a biological cell to an analysis instrument of claim 1 including a pressure measuring device connected to said sample handling unit.

7. A method of reliably and reproducibly exposing the contents of a biological cell to an analysis instrument, comprising the step of:
growing upon or applying the biological cell to a first substrate,
positioning a second substrate on top of said first substrate and the biological cell,
controllable and reproducibly applying pressure to said first substrate and said second substrate to flatten the biological cell exposing the contents of the biological cell to an analysis instrument.

8. The method of reliably and reproducibly exposing the contents of a biological cell to an analysis instrument of claim 7 wherein said step of controllable and reproducibly applying pressure to said first substrate and said second substrate comprises applying mechanical pressure to said first substrate and said second substrate.

9. The method of reliably and reproducibly exposing the contents of a biological cell to an analysis instrument of claim 7 wherein said step of controllable and reproducibly applying pressure to said first substrate and said second substrate comprises applying hydraulic pressure to said first substrate and said second substrate.

10. The method of reliably and reproducibly exposing the contents of a biological cell to an analysis instrument of claim 7 wherein said step of controllable and reproducibly applying pressure to said first substrate and said second substrate comprises applying pressure to said first substrate and said second substrate using an electrical device.

11. The method of reliably and reproducibly exposing the contents of a biological cell to an analysis instrument of claim 7 wherein said step of controllable and reproducibly applying pressure to said first substrate and said second substrate comprises applying pressure to said first substrate and said second substrate using a mechanical device or a hydraulic device or an electrical device or a combination of said mechanical device or said hydraulic device or said electrical device.
